(19) United States
(12) Patent Application Publication
(10) Pub. No.: US 2026/0165872 A1
(43) Pub. Date: Jun. 18, 2026

(54) SYSTEMS AND METHODS FOR AN IMPLANTABLE STOMA DEVICE

(71) Applicant: Davol Inc., Warwick, RI (US)

(72) Inventors: Matthew Rothberg, Atlanta, GA (US); Saisamhitha Dasari, Baltimore, MD (US); Ruofei Bu, Durham, NC (US); Anne Straits, Millersburg, OH (US)

(73) Assignee: Davol Inc., Warwick, RI (US)

(21) Appl. No.: 18/984,333

(22) Filed: Dec. 17, 2024

(51) Int. Cl. A61F 5/445; A61F 5/44

(52) U.S. Cl. CPC A61F 5/445; A61F 5/4404; A61F 2005/4455

(57) ABSTRACT

System and methods related to stoma devices are disclosed. A stoma device may be implanted in a subject and a stoma may be attached to the stoma device. The stoma may be attached to an angled outer surface of the stoma device. The stoma device may include an inflatable bladder configured to form a barrier between the stoma device and a portion of the subject underlying the inflatable bladder. The stoma device includes a body which can have a first portion comprising a first material and a second portion comprising a second material which has a different elasticity than the first material.

### FIELD

Disclosed embodiments are related to systems and methods for an implantable stoma device.

### BACKGROUND

An operation to form a stoma can include a stoma device. For example, a stoma device may be used to provide structural support for maintaining a desired position and spacing of the stoma opening from the exterior skin of a user.

### SUMMARY

In some embodiments, a stoma device comprises: a body including a channel extending through the body; a suture flange coupled to and extending out from a lower portion of the body, wherein the suture flange is configured to be attached to tissue to anchor the stoma device in a subject; and an inflatable bladder coupled to and extending out from a bottom portion of the suture flange, wherein the inflatable bladder is configured to be inflated.

In some embodiments, a method for installing a stoma device comprises: positioning a stoma device within a subject; suturing a suture flange of the stoma device to anchor the stoma device in a subject; and inflating an inflatable bladder of the stoma device to form a barrier between the suture flange and a portion of the subject's body underlying the suture flange.

In some embodiments, a stoma device comprises: a body including a channel extending through the body; a suture flange coupled to and extending out from a lower portion of the body, wherein the suture flange is configured to be attached to tissue to anchor the stoma device in a subject; and an angled outer surface coupled to the body and having a plurality of openings positioned on a perimeter of the angled outer surface, wherein the openings of the angled outer surface are configured to be attached to a stoma received in the channel.

In some embodiments, a method for installing a stoma device comprises: positioning a stoma device within a subject; suturing a suture flange of the stoma device to anchor the stoma device in the subject; and attaching the stoma to a plurality of openings formed on a perimeter of an angled outer surface of the stoma device.

In some embodiments, a stoma device comprises: a body including a shaft and a channel extending through the body, wherein at least part of the shaft is configured to extend above a skin surface of a subject when the stoma device is implanted in a subject; and a rigid seal slidingly disposed on a non-porous portion of the shaft, wherein the rigid seal is configured to be slid into contact with at least a portion of a skin of the subject adjacent to the shaft.

In some embodiments, a method for installing a stoma device comprises: positioning a stoma device within a subject, wherein positioning the stoma device within the subject includes positioning at least part of a shaft of the stoma device above a skin surface of the subject; suturing a suture flange of the stoma device to anchor the stoma device in the subject; and sliding a rigid seal along a non-porous portion of the shaft into contact with the skin surface.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows an isometric view of a stoma device according to some embodiments;

FIG. 2 shows a side view of a stoma device according to some embodiments;

FIG. 3 shows another isometric view of a stoma device with an external skin seal omitted for visualization purposes according to some embodiments;

FIG. 4 shows yet another isometric view of a portion of a stoma device according to some embodiments; and

FIG. 5 shows a bottom isometric view of a portion of a stoma device according to some embodiments;

FIG. 6 shows a top view of a stoma device according to some embodiments;

FIG. 7 shows a cross-sectional side view of a stoma device according to some embodiments;

FIG. 8 shows a stoma device arranged relative to a layer of skin according to some embodiments;

FIG. 9 shows a stoma device including a first portion made from a first material and a second portion made from a second material according to some embodiments; and

FIG. 10 shows a flow diagram related to the formation of a stoma in a subject using a stoma device according to some embodiments.

### DETAILED DESCRIPTION

An ostomy is a procedure which includes creating an opening in an abdominal wall and forming a stoma through which fluids from a bowel may exit the body of a subject. Ostomy devices, e.g., implantable stoma devices, exist to aid in the formation and function of the stoma but have several drawbacks which the inventors have recognized. For example, portions of a subject's body such as internal organs and muscles may interfere with the stoma and stoma device by contributing to tensions, agitations, or other forces that urge the stoma to retract into the body of the subject or tear the stoma. Existing stoma devices fail to adequately reduce retraction and tearing of the stoma. In some cases, retractions or tearing of the stoma require a subject to undergo surgery to adjust or reform the stoma, leading to additional cost and time expenditures for the subject and medical provider. Stoma retraction may also lead to leaking of effluent, e.g., fluids from the bowel, onto undesired locations such as skin of the subject.

In view of the above, the inventors have recognized a need for systems and methods that provide improved (e.g., more stable) anchoring of a stoma device in a subject, reduced agitation, reduction in other forces applied to the stoma which may result in retraction and/or tearing of the stoma, reduced leakage of fluids from the stoma, and/or reduced pain for the subject during and/or after implantation.

To provide improved anchoring of a stoma device to a subject, in some cases, a stoma device may include a base adapted to be fixed in the subject, for example by suturing a flange coupled to a base of the device to tissue of the subject. While this may provide improved structural support and maintenance of position and orientation of the stoma device in the subject, such an arrangement may also be prone to interference and/or adhesion from adjacent underlying tissues and/or bowels. Thus, the inventors have recognized that inflatable structure, such as an inflatable bladder, may be used to isolate the suturing flange from the other adjacent tissue and structures. For example, a stoma device may include an inflatable bladder configured to be inflated to form a barrier between a portion of the subject's body and part of the stoma device sutured to the patient.

Forming a barrier with the above noted inflatable structure may help to disperse and/or reduce forces from movement of the stoma device and portions of the subject's body, which can result from walking, standing up, exercising, or other activities by the user. For example, the inflatable bladder may flex in response to forces acting on the inflatable bladder, which may reduce forces acting on sutures connecting the stoma device to the stoma and other portions of the subject's body. Reducing these forces can reduce pain experienced by the subject as well as preventing disconnection of the stoma device from the subject. Another potential benefit associated with forming a barrier with an inflatable element is a reduction in adhesion between the subject's body and the associated portion of the stoma device. For example, the inflatable bladder may be formed with a material and/or with a geometry which reduces adhesion between the inflatable bladder and tissue of the subject. The inflatable bladder may also reduce adhesions by creating space and thereby reducing or preventing contact between the tissue of the subject and other portions of the stoma device. Reducing adhesion between the stoma device and portions of the subject's body may help to make removal of the stoma device easier, less painful, and faster. For example, a portion of the subject which is adhered to the stoma device may need to be removed or otherwise treated to be removed from contact with the stoma device, which may increase the time needed to remove the stoma device and pain experienced by the subject during removal. As referred to herein, the portions of a subject's body may include an abdominal wall of the subject.

The inflatable bladder may be inflated to transition the inflatable bladder from an uninflated state to an inflated state. Inflating the bladder may include directing fluid into the inflatable bladder, e.g., into an expandable interior volume of the inflatable bladder. The inflatable bladder may be formed with various materials or combinations of materials which are suitable to form an expandable volume, and in some cases a reversibly expandable volume, and which is suitable for implantation into a subject. The inflatable bladder may comprise an impermeable material which may resist fluid from the body of the subject from entering the interior volume of the inflatable bladder and which may hold fluid, such as a liquid or gas used to inflate the inflatable bladder, within the inflatable bladder without leaking outside the inflatable bladder. The inflatable bladder may comprise any appropriate material as the disclosure is not limited in this sense. For example, the inflatable bladder may comprise one or more of silicone, polyurethane, polyether block amide (Pebax), nylon, polyethylene terephthalate (PET), combinations of the forgoing, and/or any appropriate flexible and substantially impermeable material. The inflatable bladder can be formed in any appropriate geometry, which in some cases may include a channel extending through a central portion of the inflatable bladder. For example, the inflatable bladder may be formed in a toroidal shape where the torus includes a semi-circular, circular, and/or non-circular cross section and may enable the stoma to be passed through the central portion of the toroid and through the stoma device. Of course other non-toroidal geometries may also be used. In some cases, the stoma device may also include a plurality of inflatable bladders distributed around a perimeter of the associated flange configured to be sutured to tissue of the subject.

The Inventors have recognized benefits associated with improving the mechanical fixation of a stoma opening on an exterior opening of a stoma device. For example, in some embodiments, an exterior opening of a stoma device may be formed in an angled outer portion of the stoma with an angled outer surface formed thereon. The angled surface is adapted to be attached to a stoma. For example, attaching the stoma to the angled outer surface of the stoma device can provide structural support for the stoma and help to reduce retraction of the stoma as well as adhesion of the subject to the stoma device. Attaching the stoma to an angled surface can increase an amount of friction and retention forces applied between the stoma and the stoma device, thereby increasing an amount of force needed to retract the stoma. Further, attaching the stoma to an angled surface may decrease tension on the sutures attaching the stoma to the stoma device, which may increase the amount of force needed to retract the stoma. The angled outer portion and corresponding angled outer surface may be coupled to an upper portion of the stoma device that is configured to extend out from the skin of a subject when implanted. The stoma device may have openings formed in the angled surface and the underlying angled portion of the which can be attached to the stoma, for example by suturing the stoma to the openings. In some embodiments, the stoma device includes a channel through which the stoma passes through when implanted, and the portion of the stoma which is passed through the channel may pass through the outer opening formed in the angled outer surface and be attached to one or more portions of the angled outer surface. The stoma may be passed through an interior portion of the stoma device, including the channel. In some cases, a portion of the stoma which extends through the channel may be at least partially folded to contact the angled outer surface and may be attached to the angled outer surface. For example, the stoma may have an interior path through which the fluid exits the body, and the stoma may be folded, e.g., using an end surface of the stoma, to form an outer lip formed exterior to the interior path.

The angled outer surface of the stoma device may be formed with any appropriate angle relative to a channel extending through the stoma device as the disclosure is not limited in this sense. In some cases, the angle of the outer angled surface may be measured with respect to a longitudinal axis of the stoma device which extends through a central portion of a channel of the stoma device adapted to receive the stoma. Further, in some embodiments, a direction perpendicular to and extending out from at least a portion of the angled surface may include a first component that extends out from the longitudinal axis of the channel and a second component that extends in a direction parallel to the longitudinal axis extending from an internal opening of the channel to the outer opening of the channel. In some embodiments, the angle of the angled outer surface relative to the longitudinal axis of the channel may be less than or equal to approximately 75 degrees, 60 degrees, 45 degrees, 30 degrees, 15 degrees, and/or any other appropriate angle. The angle of the angled outer surface may also be greater than or equal to 15 degrees, 30 degrees, 45 degrees, 60 degrees, 75 degrees, and/or any other angle. Combinations of the foregoing, including angles between or equal to 15 degrees and 75 degrees are also contemplated, as well as angles above and below the ranges listed above, as the present disclosure is not limited by the angle of the angled outer surface.

The inventors have recognized that providing an exterior seal may help to shield the skin and opening in the abdominal wall of a subject adjacent to the stoma device from effluent. For example, the seal may be a rigid seal configured to slide along an exterior portion of the stoma device that is configured to extend out from a subject's skin when implanted. Thus, the seal may be configured to be moved into contact or close proximity to the skin of a subject (e.g., skin of the subject adjacent the shaft). The seal may include ergonomic features such as a recessed portion to improve the grip of a user on the seal. The seal can include a flat surface which may be formed on the bottom of the seal that is configured to be oriented towards the user's skin when the stoma device is implanted. This may help to form a flush interface between the seal and the skin of the subject. The seal being slidable along a portion of a length of the stoma device may allow the position of the seal to be adjusted, which may allow the stoma device to be implanted in in a range of skin and muscle tissue thicknesses. In some cases, a portion of a subject may be pinched between the suture flange of the stoma device and the seal, which may also help to maintain a position and orientation of the implanted stoma device.

The inventors have recognized that forming a portion of a stoma device with a flexible material may provide several potential benefits. For example, forming a portion of the stoma device which is configured to be attached to the stoma out of a flexible material may allow for easier manipulation of the flexible portion of the stoma device by a user during implantation of the stoma device, thereby allowing for faster and/or more accurate implantation of the stoma device. In such an example, another portion of the stoma device configured to be positioned below a skin surface of the subject when implanted may be formed with an appropriate structure and rigid material such that this second underlying portion of the stoma device may be more rigid than the above noted flexible portion. In one example, manipulating a flexible portion of a stoma device to orient the flexible position to receive sutures may be easier than orienting the entire stoma device to receive sutures. Decreasing the time needed to implant the stoma device may reduce the cost for the implantation procedure and may reduce the pain experienced by the subject during implantation. In some cases, an upper portion of the stoma device, for example an outer end portion, such as above noted angled outer portion, of a stoma device adapted to be attached to the stoma may be formed out of a flexible material. The outer end portion may be attached to a rigid lower portion of the stoma device that may be connected to and/or integrally formed with a fixation flange or other underlying structure. The flexible outer end portion may have a rigidity that is less than a rigidity of the other lower portion of the stoma device. For example, appropriate flexible materials may comprise any appropriate material, including but not limited to silicone, polyurethane, polyether block amide (Pebax), nylon, polyethylene terephthalate (PET), any other biocompatible polymer, and any other appropriate material exhibiting the desired amount of flexibility.

As noted above, in instances where a flexible outer portion of a stoma device is used, the stoma device may include a more rigid lower portion, e.g., stiffer than the flexible portion, that is coupled to the flexible portion to help provide structural support to the stoma device. For example, the rigid portion of the stoma device may be attached to an interior portion of the subject's body, which may help to reduce flexion of the portion of the stoma device implanted within the subject. Reducing flexion of the portion of the stoma device implanted within the subject may help to maintain a position and orientation of the stoma device. The rigid material may comprise any appropriate material, including but not limited to polypropylene, polyethylene, polyurethane, polyether block amide (Pebax), nylon, polyethylene terephthalate (PET), any other biocompatible polymer, and any other appropriate material.

As discussed herein, a moveable seal of the stoma device may be formed with any appropriate material or combination of materials. For example, the seal may be formed with any appropriate material or combination of materials including but not limited to polypropylene, polyethylene, polyurethane, polyether block amide (Pebax), nylon, polyethylene terephthalate (PET), any other biocompatible polymer, and any other appropriate material.

In some cases, the stoma device may include a cap configured to cover a portion of the stoma device. The cap may be selectively removed and attached by a user to uncover and cover the stoma device. In some cases, the cap may be removed so that a bag may be attached to the stoma to capture effluent exiting the body through the stoma. Covering the stoma with the cap may prevent effluent from exiting and/or leaking from the stoma. Accordingly, the cap may optionally be used to prevent leakage from the stoma while there is not a bag attached to the stoma to capture the effluent exiting the stoma. In some cases, the cap may be attached to the stoma device to form a dust and leak proof seal between an exterior space and the stoma.

In some embodiments, it may be desirable to reduce a potential for adhesion between a bowel and an internal channel of a stoma device. Specifically, in some embodiments, an exposed surface area of the stoma device within a channel of the stoma device may be reduced using a plurality of openings formed in a body of the stoma device that extend from an interior surface of the channel to an opposing exterior surface of a body of the stoma device. Reducing the surface area of the stoma device contacting the bowel extending through the device to form the stoma may help to reduce adhesion of the stoma device to the bowel of the subject. In some embodiments, a portion of a shaft of the stoma device include the plurality of openings formed therein.

The inventors have recognized that the improvements to systems and methods related to stoma devices as described herein can be implemented following a standard stoma implantation procedure and may provide the benefits described herein. The stoma device may be adapted to enable the stoma to be reversed, for example by disconnecting the stoma from the stoma device. As such, the stoma devices described herein may be reversible in the sense that they may be implanted and subsequently removed from the subject with minimal damage to the bowel, stoma, abdominal wall, skin and other portions of the subject's body. The systems and methods related to stoma devices as described herein may be compatible with current devices used in connection with ostomy, such as typical suturing instruments and others.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

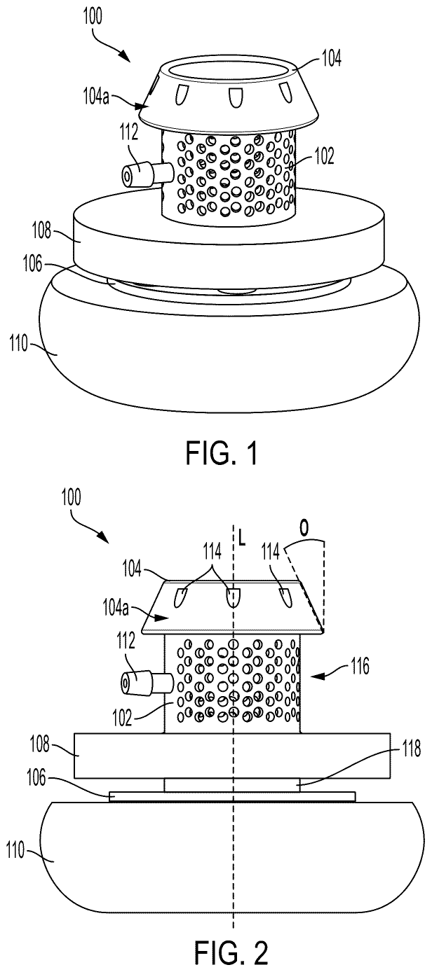

As shown in the depicted embodiment of FIG. 1, a stoma device 100 may include a body 102 according to some embodiments. A stoma attachment flange 104 may be disposed on an upper portion of the body 102 opposite from a lower portion of the body, where the stoma attachment flange 104 is adapted to be attached to a stoma according to some embodiments. For example, the stoma attachment flange 104 may comprise an outer angled surface 104a which may be coupled to the body 102 at an upper or top portion of the body 102 according to some embodiments. For example, the stoma attachment flange 104 may correspond to an angled outer portion of the stoma device 100 welded, overmolded, integrally formed with, bonded, or otherwise connected to an upper portion of the body 102. In some embodiments, the angled outer surface 104a may comprise a cylindrical and/or rounded shape. The stoma device 100 may include a suture flange 106 coupled to the body 102 and configured to be attached to tissue to anchor the stoma device 100 in a subject. The suture flange 106 may be coupled to a lower or bottom portion of the body 102 which may be opposite from the stoma attachment flange 104 according to some embodiments. In some cases, the suture flange 106 may be coupled to and extend out from a lower portion of the body 102. A seal 108, e.g., a rigid seal, may be slidingly coupled to the body 102 of the stoma device 100 according to some embodiments. For example, as shown in the figure, the rigid seal may extend at least partially, and in some embodiments completely, around a perimeter of a corresponding portion of the body 102 of the stoma device 100 (e.g., the illustrated shaft) such that the seal may be moved displaced axially along the associated portion of the body 102. In some cases, the body 102 of the stoma device 100 may include a channel extending through a shaft or other portion of the body 102. The seal 108 may be adapted to slide along at least a portion of a length of the shaft of the body extending between the stoma attachment flange 104 and suture flange 106 of the stoma device 100 according to some embodiments. In some embodiments, a channel may extend through the body 102 (e.g., the depicted shaft) as well as the opposing stoma attachment flange 104 and suture flange 106 such that opposing openings in the stoma attachment flange 104 and suture flange 106 through which the bowel used to form a stoma may pass through. The channel may be adapted to receive and/or pass the bowel through the shaft or other portion of the stoma device 100. The shaft may extend along a longitudinal axis of the stoma device 100, which in some cases may be a central axis of the stoma device 100. For example, the shaft and corresponding channel may extend along longitudinal axis “L” as shown in the depicted embodiment of FIG. 2. In the depicted embodiment of FIG. 2, longitudinal axis “L” is a central axis of the stoma device 100 and the shaft. In some embodiments, the seal 108 may be adapted to slide along the shaft of the stoma device 100 in a direction substantially parallel to the longitudinal axis “L”.

According to some embodiments, the seal 108 may be threaded onto the shaft, and as such may be rotated to axially move along the shaft via threads on the seal engaging with corresponding threads on the shaft or other portion of the body 102. In some cases, the seal 108 may include one or more detents formed on a circumference of the seal 108, which may provide an ergonomic grip for a user manipulating the seal 108. In some embodiments, the seal 108 may engage the shaft with a slip fit type connection, where moving the seal down the shaft in a direction towards the suture flange 106 may increase an amount of friction and/or engagement between the seal and the shaft. In some instances, the shaft may include a taper along the outer surface of the shaft such that an outer diameter of the shaft increases in a direction towards the suture flange 106. As discussed herein, the seal 108 may comprise a rigid material, and in some cases may be comprise the same or similar materials as a second portion 902, e.g., a rigid portion, of the stoma device 100 as discussed further with respect to the depicted embodiment of FIG. 9.

The seal 108 may be assembled onto the shaft of the stoma device 100 in any appropriate fashion as the disclosure is not limited in this sense. In some embodiments, the seal 108 may be comprised of two or more separate portions which are configured to be secured to one another around the shaft, for example using a press-fit, adhesive, or welding process. In some cases, the seal 108 may be passed over the top of the stoma device 100, e.g., over the angled outer surface 104, which may elastically deform to allow passage of the seal 108 in cases where the angled outer surface 104a is formed of a flexible material. In some embodiments, the seal 108 may be assembled or manufactured in conjunction with the shaft such that a separate process to assemble the seal 108 with the shaft of the body 102 is not necessary. For example, a seal may be assembled onto the illustrated shaft, or other portion of a body 102, prior to formation of either one of the suture flange 106 and stoma flange 104.

The stoma device 100 may comprise an inflatable bladder 110 configured to at least partially isolate and/or cushion a suture flange 106 of the stoma device 100 when inflated according to some embodiments. The inflatable bladder 110 may be coupled to the body 102, and/or the suture flange 106 according to some embodiments. In some cases, the inflatable bladder 110 may be coupled to and extend from a bottom portion of the suture flange 106 that is oriented towards an interior underlying portion of a subject (e.g., the interior of the abdominal cavity) when implanted. An opening of the channel formed in the stoma device 100 may be aligned with a corresponding opening formed in the inflatable bladder as elaborated on below.

According to some embodiments, the inflatable bladder 110 may extend outward radially and below the suture flange 106 when the inflatable bladder is in the inflated configuration. In the depicted embodiments of FIGS. 1-3, the inflatable bladder 110 is shown in an inflated configuration, where the inflatable bladder 110 may be at least partially or entirely filled with a fluid for inflating the inflatable bladder 110. The inflatable bladder 110 may be inflated with any appropriate fluid. For example, the inflatable bladder 110 may be filled with one or more of air, saline, any appropriate sterile liquid or gas, or any other appropriate fluid as the disclosure is not limited by what fluid is used to inflate the inflatable bladder 110. In some cases the inflatable bladder 110. The inflatable bladder 110 may be fluidly coupled to a flow path adapted to allow fluid to flow to and from the inflatable bladder 110, e.g., to inflate and deflate the inflatable bladder. For example, fluid may flow through the flow path into the inflatable bladder 110 to transition the inflatable bladder 110 to an inflated configuration. In another example, fluid may flow out of (e.g., be drawn from) the inflatable bladder 110 through the flow path to deflate the inflatable bladder 110. In some cases, the flow path may be formed as a channel formed in and extending through a portion of the stoma device 100. For example, the channel may be formed in and extend at least partially through one or more of the suture flange 106, the stoma flange 104, the body 102, the angled outer surface 104a, combinations thereof, and/or any other appropriate portion of the stoma device 100. Of course, instances in which a flow path is formed separately (e.g., using a separate fill port, tube, or other structure) are also contemplated.

The inflatable bladder 110 may be attached to the stoma device 100 by any appropriate attachment as the disclosure is not limited in this sense. For example, the inflatable bladder 110 may be attached to the body 102 and/or the suture flange 106 with UV cured adhesive, welding (thermal, ultrasonic, etc.), mechanical attachments, other adhesives, and/or any other appropriate type of attachment.

The stoma device 100 may include a passage 112, which in some instances may be a port or other appropriate inlet construction, which may function as an inlet for a flow path fluidly coupled to and configured to be used for inflating the inflatable bladder 110. The flow path may extend through a portion of the stoma device 100, such as wall of the body 102. In the depicted embodiments of FIGS. 1-6, the flow path extends through the body 102 from the passage 112. In some embodiments, the passage 112 may be positioned to be substantially free of contact with a subject when the stoma device 100 is implanted in the subject. In some cases, a portion of the flow path and/or passage 112 may extend through the body 102 of the stoma device 100 in a direction substantially parallel to the longitudinal axis “L” shown in the depicted embodiment of FIG. 2. The passage 112 may include any appropriate geometry adapted to enable coupling the passage 112 to a fluid source, such as a tapered portion adapted to engage a fluid source, e.g., a conduit of a fluid source, as shown in FIG. 1. In some cases, the flow path of the inflatable bladder 110 may include a valve disposed along a length and/or on an opening of the flow path that is adapted to regulate fluid flow in the flow path. For example, the flow path may include a one way valve adapted to prevent backflow of fluid from the bladder back into the flow path. In some embodiments, the flow path may extend up to a bottom surface of the suture flange 106 as shown in the depicted embodiment of FIG. 5. For example, an outlet 506 formed in on a bottom surface of the suture flange 106 adjacent to and oriented towards the bladder may be in fluid communication with the illustrated passage 112 via the flow path extending therebetween. Of course, instances in which a separately formed passage and flow path is fluidly coupled to the flexible bladder are also contemplated.

As shown in the depicted embodiment of FIG. 2, the angled outer surface 104a may include a plurality of openings 114 distributed around a perimeter of the angled outer portion 104 and/or the longitudinal axis of the device. The openings 114 may be adapted to be attached to a bowel, e.g., a stoma extending through the shaft of the body 102 of the stoma device 100. The stoma may be attached to the openings 114 by suturing, fasteners, or other types of attachments to fixate the stoma to the openings 114 according to some embodiments. In some embodiments, the openings 114 may extend through the angled surface 104a and angled portion 104 of the stoma device 100 in a direction that is substantially parallel to a longitudinal axis extending through the channel of the device. Optionally, the openings 114 may be arranged symmetrically about a central axis of the channel of the stoma device 100. The openings 114 may be formed in any appropriate size and positioning on the angled outer surface as the disclosure is not limited in this sense. In some cases, one or more of the openings 114 may have a central axis extending through the opening which is parallel to a longitudinal axis of the stoma device, e.g., the longitudinal axis “L” shown in the depicted embodiment of FIG. 2. In some further embodiments, the openings 114 may extend in one or more directions which are substantially perpendicular to the angled outer surface 104a. As discussed herein, the angled outer surface may be formed with any appropriate angle, which in some cases may be measured relative to a central axis of the stoma device 100 and/or shaft of the stoma device 100, e.g., relative to the longitudinal axis “L”. For example, the angle “O” shown in the depicted embodiment of FIG. 2 represents an angle of the angled outer surface 104a measured relative to an axis which is parallel to the axis “L”. In some cases, the angle “O” of the angled outer surface may be selected to form an appropriate amount of strain on a stoma attached to the angled outer surface. For example, too much strain on the stoma may be undesirable, as the stoma may be more susceptible to disconnection, e.g., by sutures breaking, in response to agitations and/or tensions on the stoma. The angle of the angled outer surface can be any appropriate angle as discussed above, and in some cases may be formed with an angle of about 15 degrees to about 45 degrees, though other angles as noted previously above may also be used.

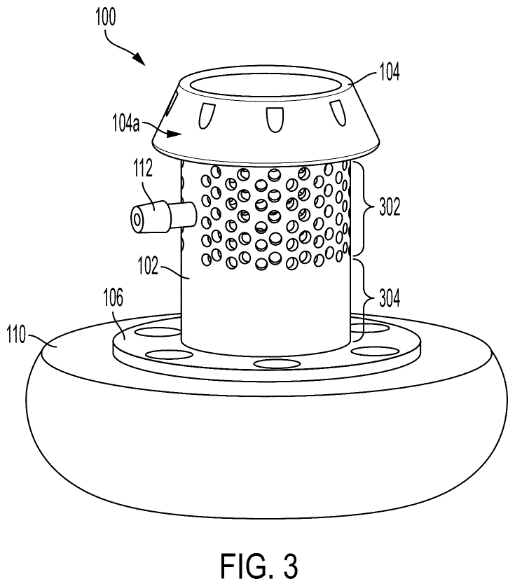

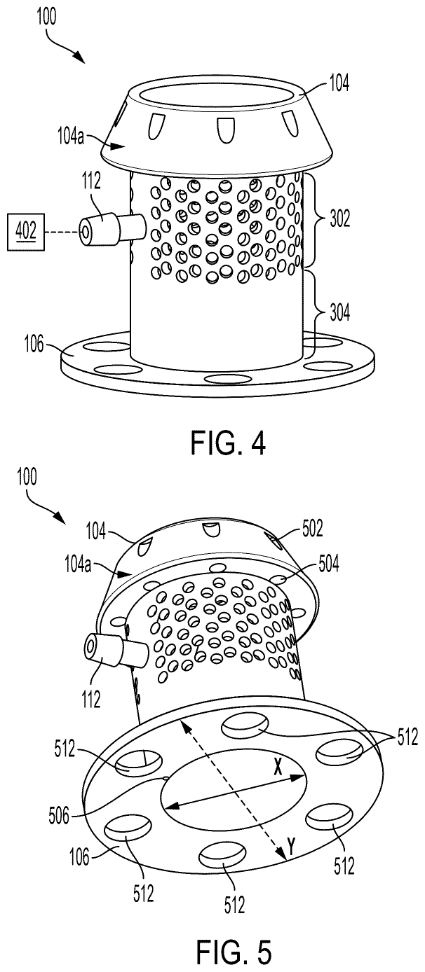

The body 102 of the stoma device 100 may include one or more openings 116 in a portion of the body extending from the suture flange 106 to the stoma attachment flange 104. The openings 116 may extend from an inner surface of the channel extending through the body 102 to an outer surface of the body 102 according to some embodiments, such as in the depicted embodiment of FIG. 2. The body 102, e.g., the shaft of the body 102, may include a porous portion and a non-porous portion. For example, the arrow denoting the openings 116 is directed toward the porous portion of the shaft and the label 118 indicates the non-porous portion of the shaft. In some embodiments, the seal is configured to be disposed on and moveable along a length of at least the non-porous portion 118 of the body 102. The depicted embodiment of FIG. 3 shows the stoma device 100 without the seal 108 and more clearly illustrates the shaft of the body 102 including a porous portion 302 and a non-porous portion 304. The depicted embodiments of FIGS. 4-5 show the stoma device 100 without the seal 108.

In some embodiments, the porous portion 302 may be positioned proximate to the angled outer surface 104a. In some embodiments, the non-porous portion 304 may be positioned proximate to and extend away from the suture flange 106. Thus, the porous portion 302 may be disposed between the non-porous portion 304 and the stoma attachment flange 104. The openings 116 may be formed in any appropriate size, shape, spacing, and/or pattern as the disclosure is not limited in this sense.

In some embodiments, the openings 116 may be formed up only on a predetermined portion of the shaft and the non-porous portion of the shaft may be adapted to slidingly engage with the seal 108. According to some embodiments, the openings 116 may be oriented such that central axes of the openings 116 are substantially perpendicular with the longitudinal axis “L” and/or central axes of the openings 114. In some embodiments, the openings 116 may be arranged as an array of circular openings, although the openings 116 may be formed in any appropriate shape, such as triangular, rectangular, any other appropriate shape, or any other appropriate combination of shapes as the disclosure is not limited in this sense. In some cases, the porous portion of the shaft may be formed on an upper portion of the shaft, and the non-porous portion of the shaft may be formed on a lower portion of the shaft, such as in the depicted embodiment of FIG. 2. In some embodiments, the flow path fluidly coupled to the inflatable bladder 110 as discussed herein may extend from the porous portion of the body 102. For example, in the depicted embodiment of FIG. 2, the passage 112 extends through the porous portion 302 and non-porous portion 304 of the body 102 and may be in fluid communication with the bladder, not depicted. However, the disclosure is not limited in this sense, as the flow path and/or passage 112 may extend from and/or be accessed via any appropriate portion of the stoma device 100 as the disclosure is not so limited.

In some embodiments, the stoma device 100 may be configured to indicate a pressure of the inflatable bladder 110, e.g., a pressure of an internal volume of the inflatable bladder 110. For example, the stoma device 100 may include or otherwise be coupled to a pressure indicator 402 configured to indicate a pressure of the inflatable bladder 110. In some cases, the stoma device 100 may include or otherwise be in fluid communication with a pressure sensor adapted to sense a pressure of the inflatable bladder and provide the sensed pressure to the pressure indicator either using a physical indicator and/or output of an electronic signal form an electronic pressure sensor. A user filling the inflatable bladder 110 with fluid may, for example, reference the pressure indicated by the pressure indicator 402 to determine when the pressure of the inflatable bladder 110 is at a suitable pressure, e.g., above a threshold pressure and/or within a suitable pressure range. In some embodiments in which an indication on the device may be desirable, the pressure indicator 402 may be formed as a dial adapted to indicate pressure, or any other pressure gauge as the disclosure is not limited in this fashion. In the depicted embodiment of FIG. 4, the pressure indicator 402 is in fluid communication with the passage 112 of the stoma device. As such, the pressure indicator may sense the pressure of the inflatable bladder 110 via the passage 112.

In some embodiments, including the depicted embodiment of FIG. 5, an inner diameter “X” of the shaft of the body 102 may be less than an outer diameter “Y” of the suture flange 106. The suture flange 106 may include one or more openings 512 adapted to be attached to a subject, e.g., tissue within the subject. The suture flange 106 may include any appropriate number of openings 512 as the disclosure is not limited in this sense. In some cases, the openings 512 may be disposed around a perimeter of the suture flange 106. The openings 512 may be formed in any appropriate geometry, including but not limited to a circular geometry. The openings 512 may extend through the suture flange 106 from an upper surface of the suture flange 106 to a bottom surface of the suture flange 106 according to some embodiments. In some embodiments, such as the depicted embodiment of FIG. 5, a flow path in fluid communication with an inflatable bladder and the passage 112 may be include an outlet 506 formed in on a bottom surface of the suture flange 106 adjacent to and oriented towards the bladder. The outlet 506 may be in sealed fluid communication with the bladder.

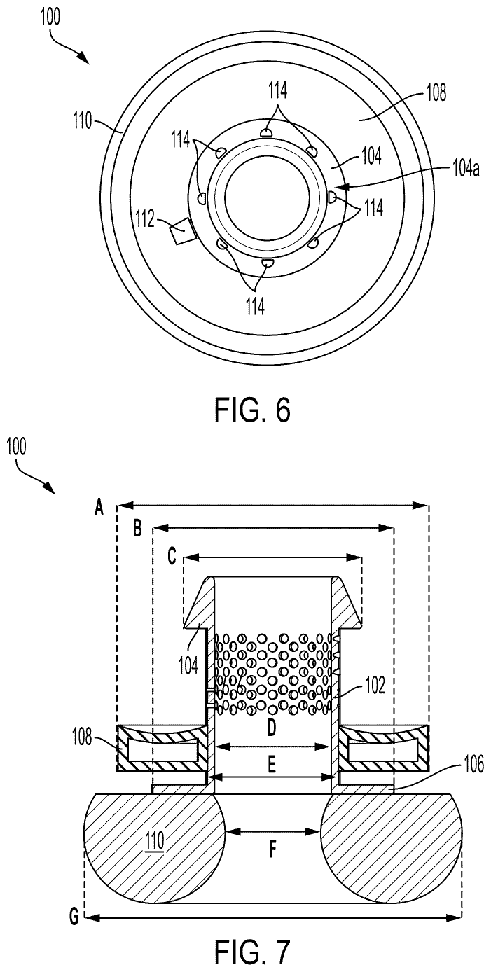

As can be best seen in the depicted embodiment of FIG. 5, the openings 114 of the angled outer surface may include an upper opening portion 502 formed on the angled outer surface 104a and a lower opening portion 504 formed on an underside of the angled outer surface 104. Specifically, the openings 114 may extend vertically through the stoma attachment flange 104 in a direction that is at least partially, and in some embodiments substantially, parallel to a direction of the longitudinal axis of the body 102 of the stoma device 100. As best seen in the depicted embodiment of FIG. 6, the openings 114 may have an at least partially semi-circular cross-section according to some embodiments.

The openings 114 on the angled outer surface 104a and the openings 512 on the suture flange 106 may be formed with any appropriate dimensions as the disclosure is not limited in this sense. In some cases, the openings 114, 512 may be formed with dimensions suitable for suturing the stoma device 100 to the tissue of the subject and/or the stoma. For example, the openings 114 and the openings 512 may be formed with diameters ranging from about 0.125 mm to about 1 mm, or any other appropriate diameters. In some cases, 3-0 polyglactin sutures may be used to suture the stoma device 100 to the tissue of the subject and/or the stoma, although any appropriate sutures with any appropriate size and/or material may be used as the disclosure is not limited in this sense.

The depicted embodiment of FIG. 7 shows a cross-sectional side view of a stoma device 100 with the inflatable bladder 110 in an inflated configuration. FIG. 7 includes distances “A”, “B”, “C”, “D”, “E”, “F”, and “G” to illustrate various dimensions of the stoma device 100 according to some embodiments. “A” may represent an outer diameter of the seal 108. “B” may represent an outer diameter of the suture flange 106. “C” may represent an outer diameter of the angled outer surface 104. “D” may represent an inner diameter of the shaft of the body 102. In some cases, “D” may also represent the inner diameter of the angled outer surface 104a. The inner diameter of the shaft and the outer diameter of the angled outer surface 104a may be selected in some cases such that the stoma device 100 may be compatible with typical ostomy covers, bags, containers, conduits, etc. In some cases, the inner diameter of the shaft of the body 102 and/or the angled outer surface may range from about 10 mm to about 25 mm. In some cases, the outer diameter of the angled outer surface may range from about 40 mm to about 50 mm. “E” may represent an outer diameter of the shaft of the body 102. “F” may represent an inner diameter of the inflatable bladder 110 in the inflated configuration. “G” may represent an outer diameter of the inflatable bladder 110 in the inflated configuration. It will be appreciated that the depicted embodiment of FIG. 7 depicts the various dimensions in relation to one another according to some embodiments, for example, the inflatable bladder 110 may have a greater outer diameter in the inflated configuration than the outer diameter of the suture flanger 106.

In some embodiments, the inflatable bladder 110 may have an outer diameter in a deflated configuration such that the inflatable bladder 110 does not interfere with attaching the suture flange 106 to the tissue of the subject. For example, the inflatable bladder 110 may have an outer diameter in the deflated configuration which is lesser than a diameter of the openings 512 on the suture flange 106. In the inflated configuration, the inflatable bladder 110 may have a greater outer diameter, e.g., distance “G”, than the outer diameter of the suture flange 106, e.g., distance “B” according to some embodiments. In some cases, the inflatable bladder 110 may extend beyond the suture flange 106, e.g., in a radial direction, by about 0.1 inches to about 1 inch, or any other appropriate distance as the disclosure is not limited in this sense. In some embodiments, the inflatable bladder 110 may have a thickness of about 10 mm inches to about 25 mm, or any other appropriate thickness as the disclosure is not limited in this sense.

As best seen in the depicted embodiment of FIG. 7, the seal 108 may include an annular recessed portion, e.g., on an upper surface of the seal 108. The annular recessed portion may improve the grip of a user on the seal 108, e.g., while sliding or otherwise manipulating the seal 108 on the stoma device 100. A lower surface of the seal 108 may be flat, which may help to create a flush surface of contact between the seal 108 and an underlying skin surface. However, the Inventors have contemplated forming the lower surface of the seal 108 with a curved or otherwise not entirely flat surface as the disclosure is not limited in this sense.

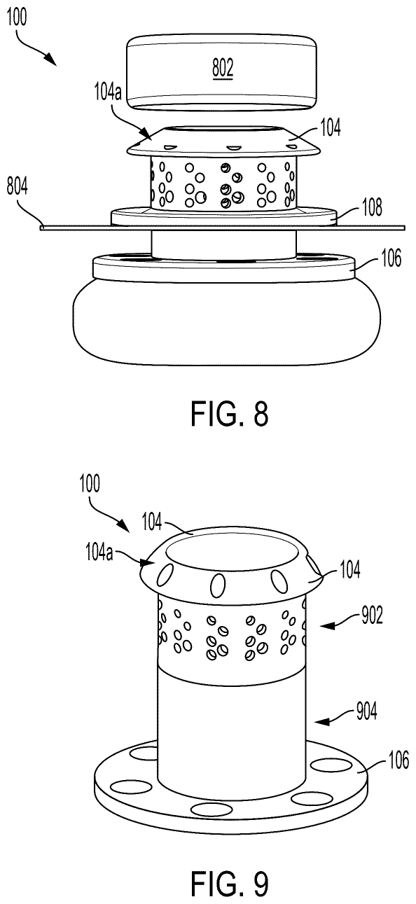

The depicted embodiment of FIG. 8 shows the stoma device with a cap 802 which may be adapted to cover a portion of the stoma device 100. For example, the cap 802 may be positioned over the angled outer surface 104a to cover the angled outer surface 104, the shaft of the body 102, and the stoma (not depicted in FIG. 8) to prevent or reduce fluid from exiting the stoma. The cap 802 may also help to prevent debris and bacteria from entering the stoma, thereby helping to prevent infection and/or clogging of the stoma. The depicted embodiment of FIG. 8 also depicts a layer of skin 804 of a subject disposed adjacent to the stoma device 100. As shown in the depicted embodiment of FIG. 8, the seal 108 may be positioned, e.g., slid along the shaft of the stoma device 100, into contact with the skin layer 804. The stoma device 100 may extend at least partially above the skin layer 804 when implanted in the subject. In some cases, a height of the portion of the stoma device 100 which extends above skin 804 may range from about 10 mm to about 50 mm, or any other appropriate height as the disclosure is not limited in this fashion. The stoma device 100 may extend below the skin layer 804 when implanted in the subject, which may depend on a thickness of the abdominal wall of the subject according to some embodiments. For example, the stoma device 100 may extend below the skin layer 804 at a depth ranging from about 20 mm to about 65 mm, or any other appropriate depth as the disclosure is not limited in this sense.

The depicted embodiment of FIG. 9 shows a stoma device 100 with a body having a first portion 902 and a second portion 904. In some embodiments, the first portion 902 of the body may be formed with a first material and the second portion 904 of the body may be formed with a second material which is more rigid than the first material. In some cases, the first portion 902 may comprise one or more of the angled outer surface 104a, the stoma attachment flange 104, the porous portion of the shaft, the passage 112 and/or any other appropriate portion of the stoma device 100. In some embodiments, the entire shaft may be formed as part of the first portion 902, and accordingly may be formed with the first material. In some cases, the second portion 904 may comprise one or more of the suture flange 106, the non-porous portion of the shaft, the passage 112 and/or any other appropriate portion of the stoma device 100. Depending on the embodiment, the seal 108 may be adapted to slide along the first portion 902 and/or the second portion 904. The first portion 902 may be coupled to the second portion 904 using additive manufacturing techniques, e.g., 3D printing, overmolding, welding, and any other appropriate type of attachment to couple the first portion 902 to the second portion 904 as the disclosure is not limited in this sense.

The first portion 902 and the second portion 904 may have different elasticities and/or hardnesses according to some embodiments. In some cases, the differences in elasticities and hardnesses of the first and second portions may be measured by a factor. For example, the hardness of the second portion to the hardness of the first portion may be a factor ranging between about 2:1 to about 5:1 measured using a Shore A durometer. That is, the hardness of the second portion may be twice as much to five times as much as the hardness of the first portion measured using a Shore A durometer. In some cases, the factor of hardness of the second portion to the first portion may preferably be about 3.5:1.

The second portion 904 may be formed with any appropriate height, e.g., measured from the bottom surface of the suture flange 106 to the upper-most surface of the second portion 904, as the disclosure is not limited in this sense. For example, the second portion 904 may have a height ranging from about 10 mm to about 75 mm, or any other appropriate height. In some cases, the second portion 904 may have a height ranging from about 25 mm to about 45 mm, or any other appropriate height.

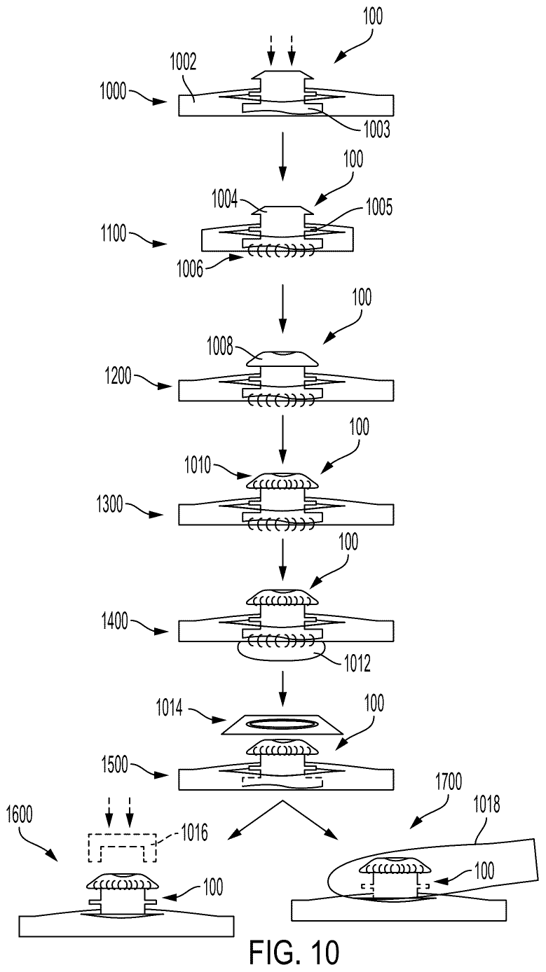

As discussed above, the systems and methods described herein may be implemented following a standard stoma implantation procedure. A method of installing a stoma device will be described with respect to FIG. 10. An incision may be formed in the skin 1002 of a subject. At 1000, a stoma device 100 may be inserted from an external space through the incision into an interior space of the subject's body below the skin 1002. As shown in 1000, another portion of the stoma device 100 may be outside of the subject, e.g., above the skin 1002. A suture flange 1003 of the stoma device 100 may be within the space below the skin, and may be attached to tissue within the subject, e.g., using sutures 1006 at 1100. A seal 1005 may be moved into contact with the skin 1002. A stoma 1008 may be formed using a bowel of the subject, and the stoma 1008 may be passed through the stoma device 100 at 1200. At 1300, the stoma 1008 may be attached to the stoma device 100, e.g., to openings of an outer angled surface 1004 of the stoma device 100 using sutures 1010. At 1400, an inflatable bladder 1012 of the stoma device 100 may be inflated, which may form a barrier between one or more portions of the stoma device 100, e.g., a body and/or the suture flange 1003 of the stoma device 100, and portions of the subject underlying the suture flange 1003 when the stoma device 100 is implanted in the subject. At 1500, a wafer 1014 may be positioned around the stoma device 100, which may include passing the wafer 1014 over the stoma device 100 and into contact with the skin 1002 to cover and/or seal the incision. As shown at 1600, a cap 1016 may optionally be used to cover at least a portion of the stoma device 100, e.g., to block fluid from existing the stoma 1008. As shown at 1700, a bag 1018, or any other appropriate container or receptacle, may optionally be coupled to the stoma device 100 and/or stoma 1008 to receive fluid exiting the stoma 1008.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A stoma device comprising:
a body including a channel extending through the body;
a suture flange coupled to and extending out from a lower portion of the body, wherein the suture flange is configured to be attached to tissue to anchor the stoma device in a subject; and
an inflatable bladder coupled to and extending out from a bottom portion of the suture flange, wherein the inflatable bladder is configured to be inflated.

2. The stoma device of claim 1, further comprising a stoma attachment flange disposed on an upper portion of the body opposite from the lower portion of the body, wherein the stoma attachment flange is configured to be attached to a stoma.

3. The stoma device of claim 2, further comprising a plurality of openings extending from an interior surface of the channel to an exterior surface of the body.

4. The stoma device of claim 2, wherein the channel is configured to pass the stoma from an internal opening of the channel configured to be positioned within the subject to an external opening of the channel.

5. The stoma device of claim 2, wherein the stoma attachment flange is angled relative to a longitudinal axis of the channel.

6. The stoma device of claim 1, further comprising a flow path in fluid communication with the inflatable bladder and configured to flow a fluid from a fluid source to the inflatable bladder.

7. The stoma device of claim 6, wherein the flow path extends at least partially through the body of the stoma device.

8. The stoma device of claim 6, wherein the fluid is at least one of a gas or liquid.

9. The stoma device of claim 7, further comprising an inlet to the flow path extending from the body of the stoma device.

10. The stoma device of claim 1, wherein the inflatable bladder is configured to form a barrier between the suture flange and a portion of the subject's body underlying the suture flange when the stoma device is implanted in the subject.

11. The stoma device of claim 10, wherein the portion of the subject's body includes an abdominal wall.

12. The stoma device of claim 1, wherein the suture flange extends radially from the lower portion of the body.

13. The stoma device of claim 1, wherein the inflatable bladder extends outward radially from the suture flange when the inflatable bladder is in an inflated configuration.

14. A method for installing a stoma device, the method comprising:
positioning a stoma device within a subject;
suturing a suture flange of the stoma device to anchor the stoma device in a subject; and
inflating an inflatable bladder of the stoma device to form a barrier between the suture flange and a portion of the subject's body underlying the suture flange.

15. The method of claim 14, further comprising forming a stoma with a bowel.

16. The method of claim 15, further comprising attaching the stoma to the stoma device.

17. The method of claim 15, further comprising moving the stoma through an interior portion of the stoma device.

18. The method of claim 14, further comprising receiving a gas or liquid within the inflatable bladder.

19. The method of claim 14, wherein the portion of the subject's body includes an abdominal wall.

20. The method of claim 14, further comprising attaching a cap to the stoma device to form a dust-proof seal between an exterior space and the stoma.

21. A stoma device comprising:
a body including a channel extending through the body;
a suture flange coupled to and extending out from a lower portion of the body, wherein the suture flange is configured to be attached to tissue to anchor the stoma device in a subject; and
an angled outer surface coupled to the body and having a plurality of openings positioned on a perimeter of the angled outer surface,
wherein the openings of the angled outer surface are configured to be attached to a stoma received in the channel.

22-30. (canceled)

31. A method for installing a stoma device, the method comprising:
positioning a stoma device within a subject;
suturing a suture flange of the stoma device to anchor the stoma device in the subject; and
attaching the stoma to a plurality of openings formed on a perimeter of an angled outer surface of the stoma device.

32-38. (canceled)

39. A stoma device comprising:
a body including a shaft and a channel extending through the body, wherein at least part of the shaft is configured to extend above a skin surface of a subject when the stoma device is implanted in a subject; and
a rigid seal slidingly disposed on a non-porous portion of the shaft, wherein the rigid seal is configured to be slid into contact with at least a portion of a skin of the subject adjacent to the shaft.

40-52. (canceled)

53. A method for installing a stoma device, the method comprising:
positioning a stoma device within a subject, wherein positioning the stoma device within the subject includes positioning at least part of a shaft of the stoma device above a skin surface of the subject;
suturing a suture flange of the stoma device to anchor the stoma device in the subject; and
sliding a rigid seal along a non-porous portion of the shaft into contact with the skin surface.

54-62. (canceled)
